(19) United States
(12) Patent Application Publication
(10) Pub. No.: US 2010/0010356 A1
(43) Pub. Date: Jan. 14, 2010

(54) BLOOD PRESSURE MONITOR WITH REMOTE DISPLAY

(76) Inventors: Raymond Chan, Hong Kong (HK); Mun Hoong Leung, Hong Kong (HK); Li Li, Hong Kong (HK)

Correspondence Address: EAGLE IP LIMITED, 13/F, Bright Way Tower, 33 Mong Kok Road, Kowloon, HK

(21) Appl. No.: 12/170,430

(22) Filed: Jul. 9, 2008

(51) Int. Cl. A61B 5/0225

(52) U.S. Cl. 600/493; 600/490

(57) ABSTRACT

A blood pressure monitor having a wireless transmitter is disclosed. A pressure sensor measures blood pressure data from a target, and the blood pressure data is transmitted to a remote device through the wireless transmitter. In one embodiment, the remote device is a mobile phone or a PDA. An alternate blood pressure monitor having an electronic display unit displaying the instantaneous pressure inside a cuff is also disclosed. In an embodiment, the blood pressure monitor computes the blood pressure value of the target, and the user can choose the measuring mode of the blood pressure monitor.

### FIELD OF INVENTION

The present invention relates to a blood pressure monitor for measuring blood pressure at a human body.

### BACKGROUND OF INVENTION

Conventional blood pressure monitors have an air tube between the cuff and the main body, such as disclosed by Inagaki et al. in U.S. Pat. No. 6,344,025. However, such a blood pressure monitor is only able to display simple results, and it is not efficient if more detailed information is needed. Therefore there is a need to improve the current blood pressure monitor.

### SUMMARY OF INVENTION

In the light of the foregoing background, it is an object of the present invention to provide an alternate blood pressure monitor.

The blood pressure monitor according to the present invention comprises a wireless transmitter that sends the measured data from a pressure sensor to a remote device having a wireless receiver. In an embodiment, the remote device has a microprocessor that processes the measured data from the pressure sensor and a display unit that displays the processed data.

In another embodiment, the remote device is a portable device such as a mobile phone. A software program is installed in the remote device to process the received data. In one embodiment, the remote device can further forward the data to other remote devices.

In another aspect of the present invention, the blood pressure monitor has an electronic display unit displaying instantaneous pressure inside a cuff. In another embodiment, the user pre-selects the rate of deflation and the electronic display unit displays an “intended value” of the pressure inside the cuff from a plurality of parameters.

In another embodiment, the blood pressure monitor comprises a recording mechanism. A user presses a button to record the displayed value into the blood pressure monitor, and the value can be retrieved at a later time.

In one embodiment, the blood pressure monitor further comprises an algorithm to compute blood pressure values for the target. In another embodiment, the measuring mode is selectable by the user.

### BRIEF DESCRIPTION OF FIGURES

FIG. 1 is a diagram of the first embodiment of the invention when in use.

FIG. 2 is an electrical block diagram of the embodiment shown in FIG. 1.

FIG. 3 is a diagram of the second embodiment of the invention when in use.

FIG. 4 is an electrical block diagram of the embodiment shown in FIG. 3.

FIG. 5 is a data packet that is transmitted according to an exemplary embodiment.

FIG. 6 is a flow chart of the algorithm that the remote device processes the received data packets in an exemplary embodiment.

### DETAILED DESCRIPTION OF THE PREFERRED EMBODIMENTS

As used herein and in the claims, “comprising” means including the following elements but not excluding others.

As used herein and in the claims, “connect” refers to electrical coupling or connection either directly or indirectly via one or more electrical means unless otherwise stated.

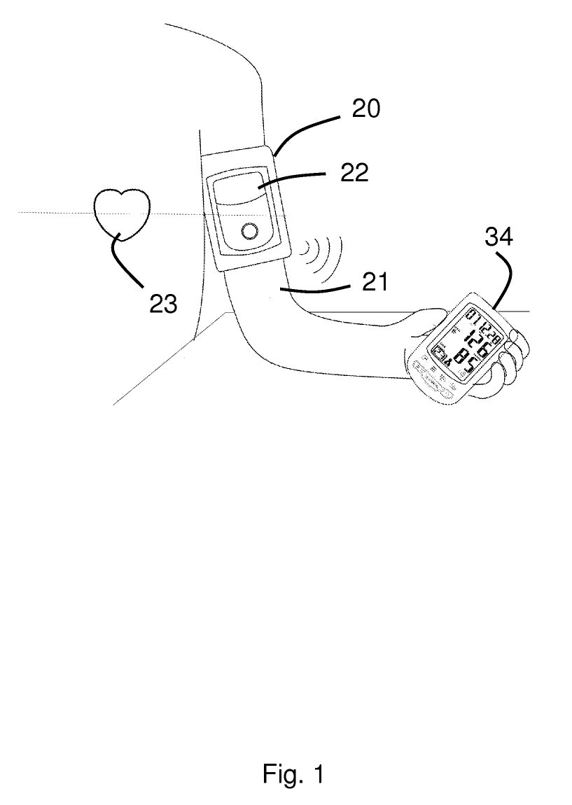

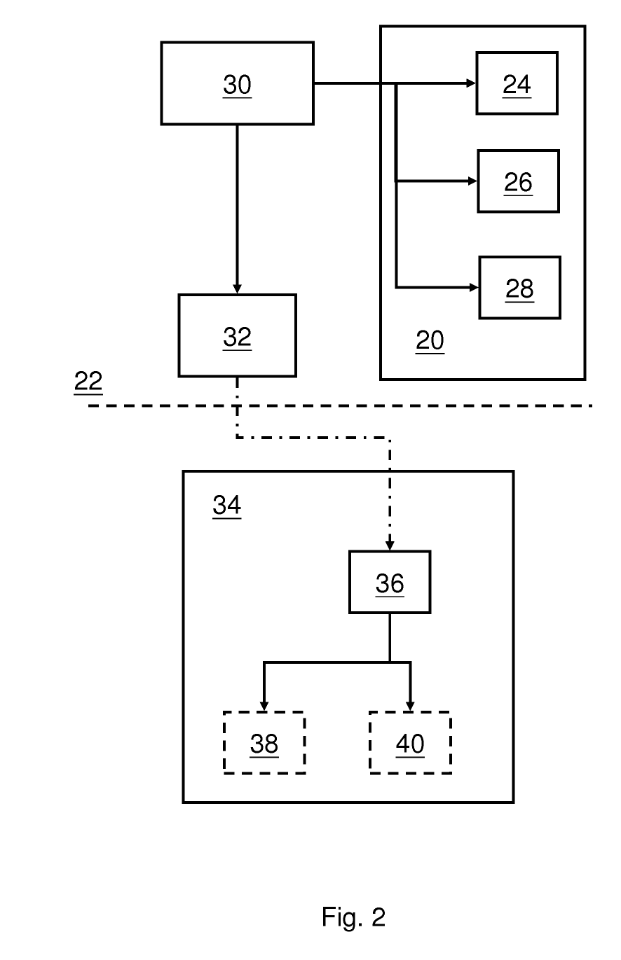

Referring to FIGS. 1 and 2, the first embodiment of the present invention is a blood pressure monitor comprising two parts. The first part includes a cuff 20 and a main body 22 attached to the cuff 20. A number of pumps 24 and valves 26 are disposed at the surface of the cuff 20, connected to the main body 20. A pressure sensor 28 is fitted inside the cuff 28. A microcontroller unit 30 (MCU) is provided inside the main body 22, and is connected to the pressure sensor 28. Inside the main body 22 is also a wireless transmitter 32 connected to the MCU 30. The second part is a remote device 34 that is physically separate from the first part. A wireless receiver 36 is disposed inside the remote device. An algorithm is programmed in the MCU 30 to control the wireless transmitter 32 to send a signal to the remote device.

Before operation, the cuff 20 is first wrapped around a target's forearm 21 or wrist, with the forearm 21 or wrist raised to the same height level as target's heart 23. When the blood pressure monitor is activated, the MCU 30 will control the pump 24 to inflate the cuff 20 to a pre-determined pressure level above the systolic pressure of a normal target, and is called high level here and throughout the specification. The pressure inside the cuff 20 then slowly decreases through opening of the valves 26 controlled by the MCU 30 until the pressure is decreased to a pre-determined level that is below the diastolic pressure of a normal target, which is called low level. The pressure sensor 28 monitors the pressure inside the cuff 20 throughout the period.

When the pressure inside the cuff 20 is at high level, blood flow in blood vessels of the forearm or wrist is completely stopped. As a result, the pressure is stable in this situation. As the pressure is slowly decreased, blood flow starts to occur, but the flow is still restricted by the cuff 20. Blood flowing through the blood vessels induces a rhythmatic contraction and dilation pattern at the blood vessels. The contraction and dilation of the blood vessels changes the pressure inside the cuff 20 and the pressure change is detected by the pressure sensor 28. This pattern continues until the pressure inside the cuff is at low level, such that blood flow is unimpeded and the pressure inside the cuff is stable again. The pressure data is then processed with an algorithm in the MCU 30 to compute the systolic and diastolic pressures of the target. The pressure data, along with the computed systolic and diastolic pressure values of the target, is then transmitted to the remote device 34 through the wireless transmitter 32. In one embodiment, the wireless transmitter 32 is a 433 MHz or 868 MHz radio frequency (RF) transmitter.

In one embodiment, the remote device 34 comprises a microprocessor 38 and a display unit 40. After receiving the pressure data from the main body 22, the microprocessor 38 processes the data and the display unit 40 displays the desired information, for example the systolic/diastolic values or the whole pressure graph. In one embodiment, the remote device 34 also has an option to further forward the pressure data to other remote devices through wired or wireless transmission protocols. In another embodiment, the remote device 34 is a portable device such as a mobile phone or a personal digital assistant (PDA).

This invention enables the data to be sent to another device that is more powerful than the MCU 30 inside conventional blood pressure monitors. As a result, one can extract the most information from the data check each and every time, without the need to constantly change the blood pressure monitor to an updated model. For example, the measured data may indicate what diseases the target may have. A conventional blood pressure monitor can only display simple information such as systolic/diastolic pressure, while the blood pressure monitor in this invention can forward the information to a computer so that doctors can investigate the complete pattern of pressure change.

This invention also eliminates the need of a display unit 40 at the main body 22. The pressure data is forwarded to the remote device then computed for the systolic and diastolic values, and is then displayed or used for other purposes. Using wireless transmission also removes the limit of the size of the remote device 34 and the relative distance between the main body 22 and the remote device 34. For example, the remote device 34 can be a personal computer on the desk of the user having a monitor of 21 inches, or can be a mobile phone of an immediate family member having a display of 2 inches, but located in another city. Furthermore, eliminating the display unit 40 at the main body 22 reduces the weight of the main body 22 so that the blood pressure monitor is more convenient to carry around. The target also feels more comfortable when wrapped inside the cuff 20 as the frictional force that acts on the target, which is proportional of the weight of the blood pressure monitor, is reduced.

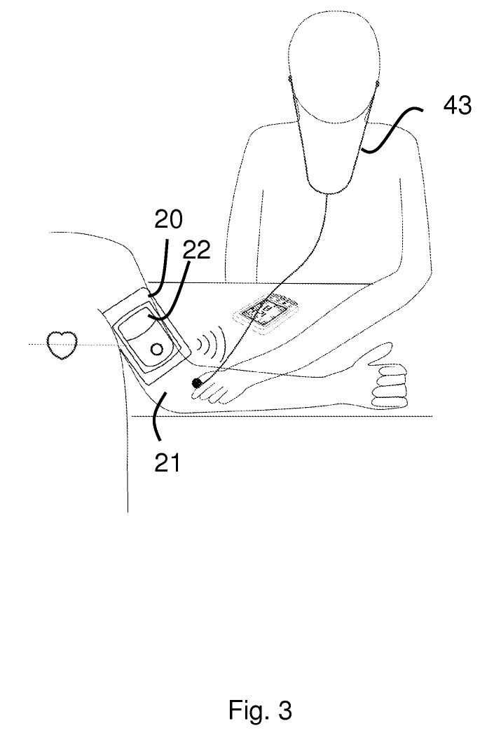

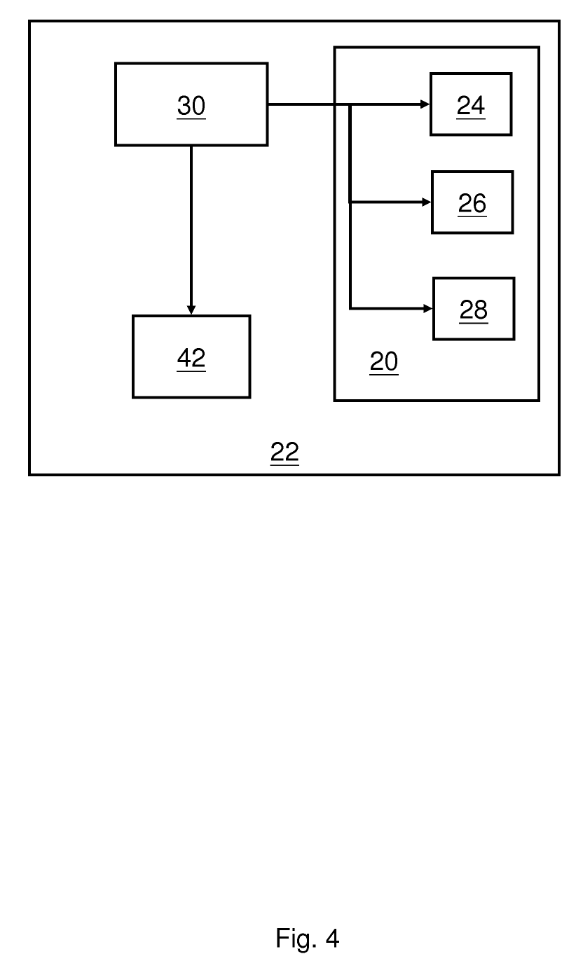

The second embodiment of the present invention as shown in FIGS. 3 and 4, is a blood pressure monitor comprising a cuff 20, a number of pumps 24 and valves 26, an electronic display unit 42, a pressure sensor 28 fitted inside the cuff 20 and a MCU 30. The MCU 30 is connected to the pumps 24 and valves 26, the electronic display unit 42 and the pressure sensor 28.

When the blood pressure monitor is activated, the pressure sensor 28 monitors the pressure inside the cuff 20 continuously, and the instantaneous pressure value is displayed on the electronic display unit 42. A user inserts a stethoscope 43 under the cuff 20 to determine the systolic and diastolic pressure values by listening to the Korotkoff sound of a target during the deflation of the cuff 20. When the user first hears the Korotkoff sound of the target, the user records the value displayed on the electronic display unit 42 at that instant as the systolic pressure. When the Korotkoff sound is no longer heard, the pressure value at that instant is recorded as the diastolic pressure of the target. In one embodiment, the user can also specify the pressure at high level and low level and also the rate of deflating of the cuff 20.

The instantaneous pressure value may fluctuate as the pressure inside the cuff 20 is affected by blood flow inside blood vessels. The user may be unable to decide which value should he record if the pressure is fluctuating and that the result may be inaccurate. In one embodiment, instead of displaying the instantaneous pressure inside the cuff 20, the user sets the high level, the low level and the time used to reduce the pressure from the high level to the low level. The electronic display unit 42 then outputs a pressure level calculated by the MCU 30 using a linear equation with the above parameters. This configuration displays the “intended” pressure inside the cuff 20, therefore the user always sees this value dropping constantly and not frequently fluctuating, making the user easier to take the reading.

In one embodiment, the blood pressure monitor further comprises a recording mechanism. When the user hears the Korotkoff sound, the user presses a save button so that the displayed value at that moment is saved inside the blood pressure monitor as the systolic pressure. The same applies for diastolic pressure. In this configuration, the user does not need to write down the pressure values during blood pressure check and can be more concentrated in the information from the stethoscope. In one embodiment, the recording mechanism is an external device connected to the main body 22 through wired or wireless communication protocols.

In one embodiment, the MCU 30 of the blood pressure monitor is further programmed to compute the pressure values of the target. In another embodiment, the mode of measurement is selectable by the user. The options are auscultatory (by listening to Korotkoff sound through stethoscope), oscillometric (detecting the change in pressure inside the cuff), or both. A user is able to compare the result between two methods when both methods are selected.

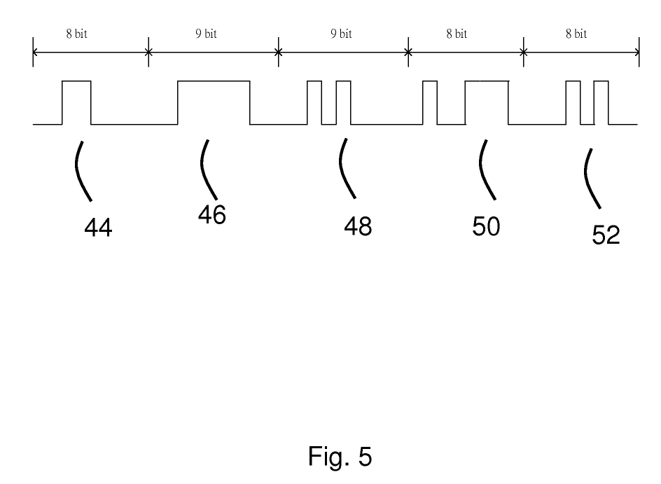

Referring now to FIG. 5, the signal transmitted from the wireless transmitter 32 comprises many data packets, with each data packet beginning with a start bit 44, which is 8-bit in length, followed by a 9-bit systolic blood pressure (SBP) value 46, a 9-bit diastolic blood pressure (DBP) value 48 and a 8-bit pulse rate value 50, collectively known as data values. The 9-bit value is an unsigned integer corresponding to a value of zero to 511, which is enough for storing blood pressure values. An 8-bit check bit 52 follows the data values before another start bit 44 is transmitted again. Data is transmitted at a rate of 200 to 500 Hz, corresponding to 2-5 ms for every bit. The wireless transmitter 32 repeats the transmitting process 8 to 12 times for the each measurement of cuff pressure to ensure the data is safely transmitted to the remote device 34. Afterwards, another measurement is taken and another set of data packets are transmitted. Typically the time between the last data packet of the first measurement and the first data packet of the second measurement is much longer than the time between two data packets in a single measurement.

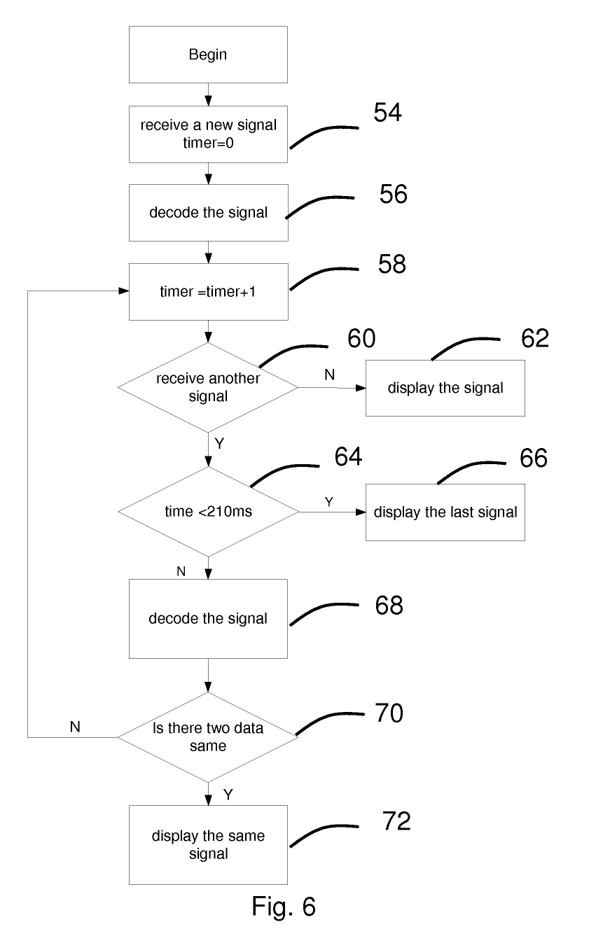

Referring to FIG. 6, the algorithm implemented at the remote device 34 to process the transmitted data is shown. First, the timer is initialized to zero (step 54). After the first data packet is decoded (step 56), the timer is incremented (step 58) each time it receives another data packet. The remote device 34 will check if another data packet is received (step 60). If the remote device 34 does not receive another data packet, it means the whole measurement process has finished, and the last received data values will be displayed (step 62). If the remote device 34 receives another data packet within the time period, it will check if the time between the two received data packets is more than 210 ms, which is (8+9+9+8+8=42 bits times 5 ms/bit) (step 64). If the remote device 34 receives two data packets within 210 ms, the second data packet is deemed to contain the same content as the first one, and the last received data values are displayed (step 66). Otherwise, the data packet is decoded (step 68) to see if the decoded data values are the same between the new data packet and the last data packet (step 70). If the two data values are the same, the remote device 34 then displays the data values (step 72). If the two data values are different, the remote device 34 will receive a third data packet. Only when two consecutive data packets are the same will the data values be displayed. If the third data packet differs in value from the second packet, the process is repeated until two consecutive data packets having the same data values are received.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the blood pressure monitor in the second embodiment can also comprise a wireless transmitter 32 to transmit signal to other remote devices 34, or the blood pressure monitor in the first embodiment can also display the instantaneous pressure at the remote device 34.

In one embodiment, the transmitted data is encrypted with known algorithms such that unauthorized users are unable to obtain the data.

In one embodiment, other information can also be displayed on the display unit 40 or electronic display unit 42, such as time, temperature, or other physiological information such as heartbeat rate. In one embodiment, the time information is sent with the measured data as a time stamp.

In one embodiment, the blood pressure monitor is calibrated by an external device. In another embodiment, the blood pressure monitor comprises a self-calibrating mechanism.

It is clear that the wireless communication protocol is not limited to 433 MHz or 866 MHz RF. Other possible protocols are 315 MHz RF, Bluetooth, Wi-Fi, or infra-red.

What is claimed is:

1. A blood pressure monitor comprising:
a) a cuff;
b) at least one pump disposed on a surface of said cuff;
c) at least one valve disposed on a surface of said cuff;
d) a pressure sensor fitted inside said cuff to measure pressure inside said cuff;
e) a microcontroller unit connected to said at least one pump, said at least one valve and said pressure sensor;
f) a wireless transmitter connected to said microcontroller unit; and
g) a remote device physically separate from said cuff; said remote device comprising a wireless receiver;
wherein said wireless transmitter transmits blood pressure data measured from said pressure sensor to said remote device.

2. The blood pressure monitor according to claim 1, wherein said remote device further comprises a display unit.

3. The blood pressure monitor according to claim 1, wherein said remote device is a mobile phone.

4. The blood pressure monitor according to claim 1, wherein said remote device is a personal digital assistant.

5. The blood pressure monitor according to claim 1, further comprising a display unit attached to said cuff.

6. The blood pressure monitor according to claim 1, wherein said remote device is configured to forward said blood pressure data to other remote devices.

7. The blood pressure monitor according to claim 1, wherein said remote device comprises a microprocessor to process said blood pressure data.

8. A blood pressure monitor comprising:
a) a cuff;
b) at least one pump disposed on a surface of said cuff;
c) at least one valve disposed on a surface of said cuff;
d) a pressure sensor fitted inside said cuff to measure pressure inside said cuff;
e) a microcontroller unit connected to said at least one pump, said at least one valve and said pressure sensor;
wherein an instantaneous pressure value is displayed on said electronic display unit such that a user can use a stethoscope for measuring blood pressure of a target by referring to said displayed pressure value.

9. The blood pressure monitor according to claim 8, wherein said blood pressure monitor does not have a systolic and diastolic sensor.

10. The blood pressure monitor according to claim 8, wherein said microcontroller unit computes blood pressure values of said target from said blood pressure data.

11. The blood pressure monitor according to claim 8, wherein said displayed pressure value is an instantaneous pressure value inside said cuff.

12. The blood pressure monitor according to claim 8, wherein said displayed pressure value is calculated by said microcontroller unit from at least one parameter.

13. The blood pressure monitor according to claim 12, wherein said at least one parameter is a high level, a low level and a time for said cuff to deflate from said high level to said low level.

14. The blood pressure monitor according to claim 8, further comprising a recording mechanism for said user to record said displayed value when said recording mechanism is activated.

15. The blood pressure monitor according to claim 14, wherein said recording mechanism comprises an external device connected to said microcontroller unit.

16. The blood pressure monitor according to claim 8, wherein a measuring mode of said blood pressure monitor is selectable by said user.

17. The blood pressure monitor according to claim 16, wherein said measuring mode is selected from a group consisting of auscultatory, oscillometric and both.

18. The blood pressure monitor according to claim 1, wherein said wireless transmitter transmits said blood pressure data to said remote device by a data packet consisting of a start bit, a check bit and data values consisting of a systolic blood pressure value, a diastolic blood pressure value and a pulse rate value.

19. The blood pressure monitor according to claim 18, wherein said start bit, said pulse rate value and said check bit are of 8-bit length, and said systolic blood pressure value and said diastolic blood pressure value are of 9-bit length.

20. The blood pressure monitor according to claim 18, wherein said remote device only displays said systolic blood pressure value, and said data values when two consecutive data packets received by said remote device have identical content.
